(19) United States
(12) Patent Application Publication
(10) Pub. No.: US 2011/0245901 A1
(43) Pub. Date: Oct. 6, 2011

(54) ICE PACK

(76) Inventors: Mei-Jiao Huang, Banchiau City (TW); Zheng-Yao Lin, Banchiau City (TW)

(21) Appl. No.: 12/897,387

(22) Filed: Oct. 4, 2010

(30) Foreign Application Priority Data
Apr. 2, 2010 (TW) 099205896

(51) Int. Cl. A61F 7/10

(52) U.S. Cl. 607/112; 607/114

(57) ABSTRACT

The present invention discloses an ice pack including a main body. A center of the main body is a bag fixing part and two ends of the bag fixing part are extended respectively with a first fastener and a second fastener which can be connected and fixed together. The bag fixing part is connected and fixed to a bag through a hook and loop fastener, an interior of the bag is filled with refrigerant and a side of the bag is provided with a hook and loop fastener, allowing the bag to be fixed on the main body. On the other hand, the other side of the bag is extended and protruded with positioning parts which are fixed at two sides of a user's nose bridge, and the bag is stably put on a forehead.

### BACKGROUND OF THE INVENTION

a) Field of the Invention

The present invention relates to an ice pack, and more particularly to an ice pack which is able to be put fixed on a human body, allowing a bag of compression to be applied on an affected part for compression and treatment, so as to achieve an effect of reducing body temperature.

b) Description of the Prior Art

A man is a homoiothermal animal and his or her core temperature is usually keeping at 37° C. When body temperature rises up due to an external factor or illness, a person will easily feel that he or she is heating up inside the body and it may damage human cells as the body temperature is too high, if the situation is severe. In particular, as rapid development of modern industries, pollution which is brought by the development is getting more and more severe, wherein increase of carbon dioxide will change a content of earth atmosphere. Therefore, the phenomenon of global warming is the most direct result, which allows temperature in all areas of world to increase year by year. A lot of zones of higher latitudes start to become hot in weather, not mentioning some tropical or subtropical zones.

In addition to the abovementioned weather change, sometimes when the human body suffers from deceases or sports injuries, the body temperature will increase, as well. For example, when the human body is infected by pathogens, an immunization system in the body will activate white blood cells to fight with the enemies and stimulate lymphocytes to produce antibodies to destroy the enemies, in collaboration with the white blood cells. After the pathogens have fought with the white blood cells, remains will release a substance called thermogen which will follow with blood circulation to hypothalamus to increase the body temperature and people will get a fever. Moreover, when people is exercising, as an amount of accumulation by repeatedly exerting a force exceeds adaptability of the body, muscles will not be able to withstand; or if gestures of exercising are inappropriate, the muscles can be infected, which will in turn allow the infected part to swell and heat up locally. The reason of this physiological reaction to appear lies in that the human is provided with a function of automatically protecting injured tissues and repairing the injured part.

All the abovementioned are the factors which may cause the increase of human body temperature and if the body temperature cannot be reduced in time, then human cells may be damaged or even people may get weak to decrease a defense function of the immunization system, due to that the body temperature is too high or rises up continuously. Therefore, there are vendors who have developed a cooling tool to reduce the body temperature.

The conventional cooling tool is an improved ice pack which is a bag unit formed by adhering a waterproofing cloth layer and a rubber layer, wherein the rubber layer is inside the bag unit, the waterproofing cloth layer is at an exterior of the bag unit and the bag unit is provided with a frame having an internal thread to form an opening of water access. The bag unit that is joined with the frame is formed in an annular pleated shape and is provided additionally with a cover having an outer thread; whereas, an outer rim of the cover is provided with plural arc-shaped ribs. By that the outer thread of the cover can be screwed into the inner thread of the frame, the cover and the frame can be locked together. In addition, a surface between the cover and the frame is provided with a waterproof gasket to prevent from water seeping out and water will not seep out of the exterior part of the bag unit.

Although the abovementioned ice pack can be used to cold compress at a local position of the human body to reduce the temperature, the entire ice pack structure is not very perfect. For example, when a personal sprains or gets a fever that he or she needs the cold compression, the ice pack is normally put at the body part and is held by a hand to press, thereby keeping the ice pack to touch the body. However, this method is very inconvenient, as the action of holding and pressing the ice pack by the hand will allow the body action to be extremely inconvenient. Moreover, if a person gets a high fever that he or she needs the cold compression, some one has to be at a side to take care of him or her all the time, otherwise, the ice pack will fall out once the patient turns his or her body, causing inconvenience in usage.

Accordingly, there are still a lot of shortcomings of the abovementioned ice pack which is indeed not a perfect design and thus requires to be improved.

### SUMMARY OF THE INVENTION

The primary object of the present invention is to provide an ice pack, wherein through provision of hook and loop fasteners on a bag fixing part of a main body, the hook and loop fasteners can correspond with hook and loop fasteners of the bag, such that an effect of connecting and fixing can be achieved, and the bag can be separated with the main body to carry out a repeated operation like cleaning or freezing for use.

Another object of the present invention is to provide an ice pack, wherein through providing more than one positioning part on a surface at one side of the bag, when the bag is put on a forehead of a user, the positioning parts can be fixed at two sides of the user's nose bridge and a middle part between two eyes, thereby stably putting the bag on the forehead to reduce the body temperature.

Still another object of the present invention is to provide an ice pack, wherein through providing refrigerant inside the bag, the temperature can be kept for a long time and the ice pack can be operated and used repeatedly, thereby largely reducing a user cost.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional exploded view of an ice pack of the present invention.

FIG. 2 shows a three-dimensional schematic view of assembly of the ice pack of the present invention.

FIG. 3 shows a three-dimensional schematic view of assembly at a rear side of the ice pack of the present invention.

FIG. 4 shows a schematic view of an implementation of the ice pack of the present invention.

### DETAILED DESCRIPTION OF THE PREFERRED EMBODIMENTS

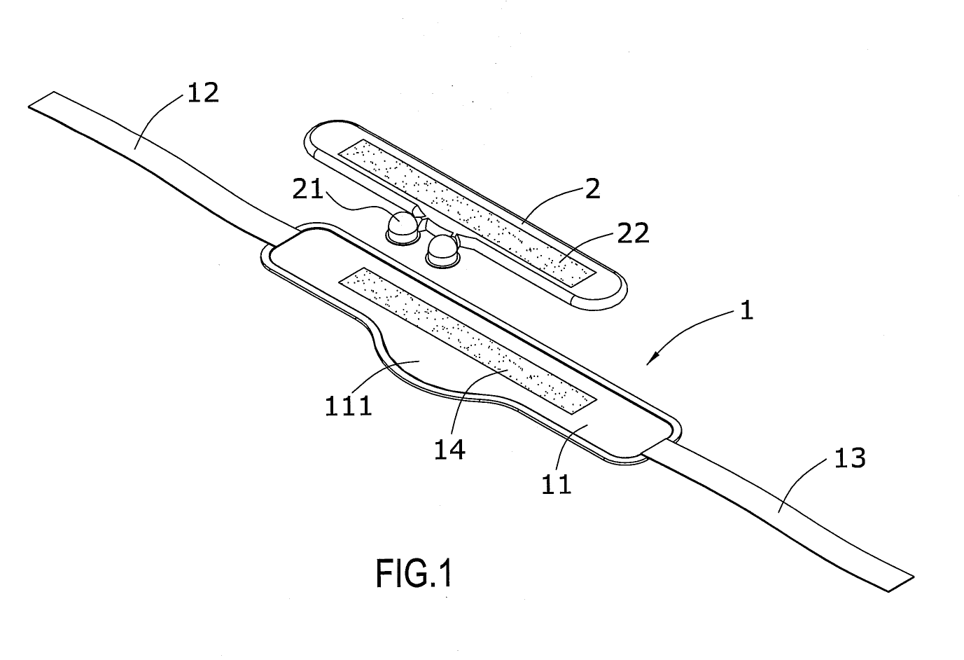

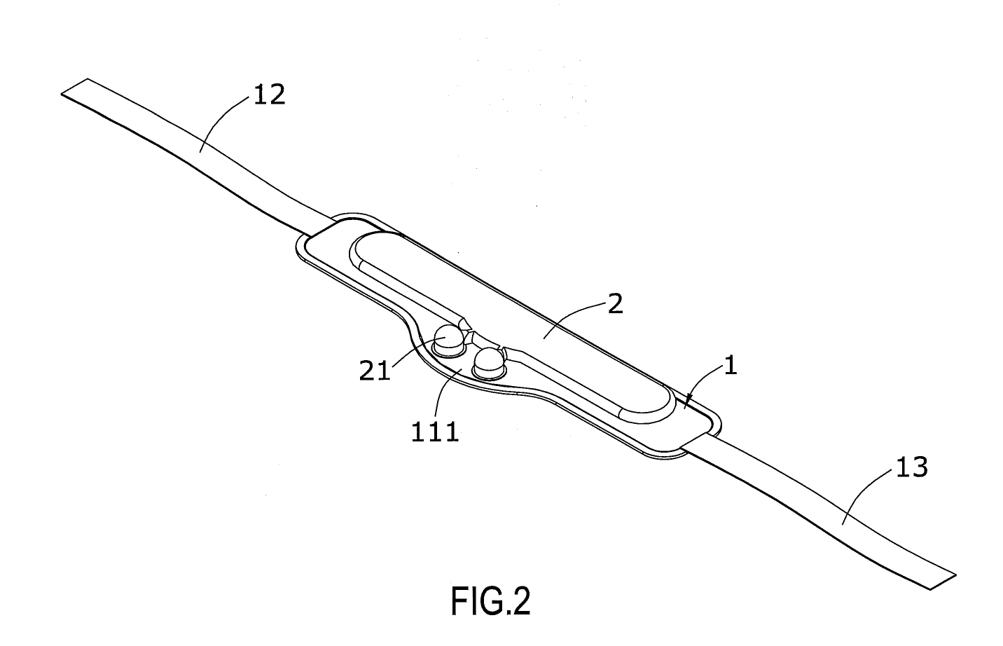

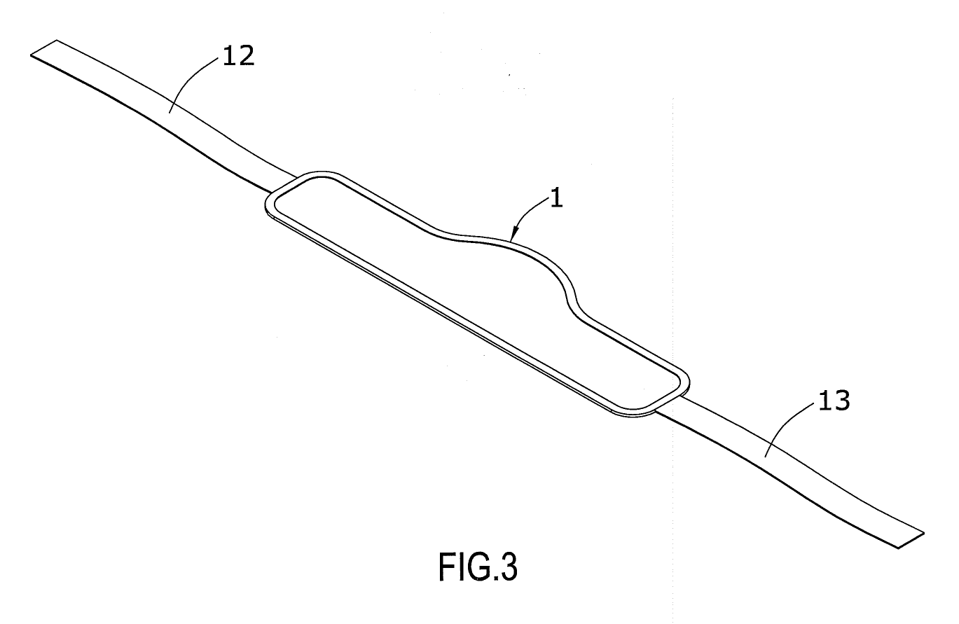

Referring to FIGS. 1 to 3, it shows a three-dimensional exploded view, a three-dimensional schematic view of assembly and a three-dimensional schematic view at another angle, of an ice pack of the present invention. As shown in the drawings, the ice pack comprises a main body 1, which is roughly a long rectangular unit and a center of which is a bag fixing part 11. A bottom end of the bag fixing part 11 is extended with an extension part 111; whereas, two ends of the bag fixing part 11 are extended respectively with a first fastener 12 and a second fastener 13. The abovementioned first fastener 12 and second fastener 13 can be hook and loop fasteners, allowing the first fastener 12 and the second fastener 13 to be connected and fixed together. A side of the bag fixing part 11 is connected and fixed with a bag 2 through a hook and loop fastener 14.

A shape of the bag 2 is about the same as that of the bag fixing part 11 and an interior of the bag 2 is filled with refrigerant through which temperature of the bag 2 can be maintained. A side of the bag 2 is provided with a hook and loop fastener 22 which corresponds to the bag fixing part 11, allowing the bag 2 to be fixed on the bag fixing part 11 of the main body 1. A bottom at a center of the bag 2 is extended and protruded with at least one positioning part 21 and in the present embodiment, a pair, although not limited by this number, of the positioning part 21 is provided.

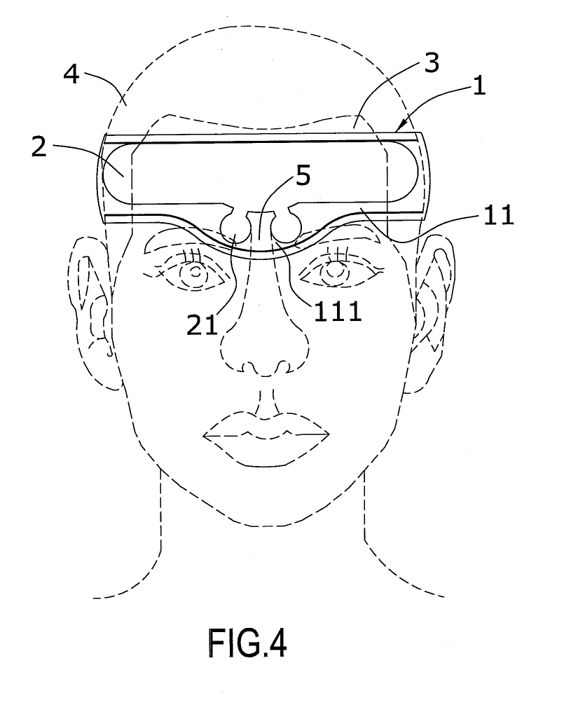

Referring to FIG. 4 at a same time, it shows a schematic view of an implementation of the ice pack the present invention. When operating and using the ice pack, the bag 2 after frozen is connected and fixed to the bag fixing part 11 of the main body 1 through the hook and loop fastener 22. At this time, the positioning parts 21 that are extended at the bottom of the bag 2 will be located on the extension part 111 at the bottom of the bag fixing part 11. Accordingly, a user can put the bag 2 on a forehead 3 of a user and at a same time, fix and latch the first fastener 12 and the second fastener 13 at a rear side of a head 4; whereas, the positioning parts 21 of the bag 2 are fixed at two sides of a nose bridge 5 of the user and located between two eyes. Through provision of the positioning parts 21, the bag 2 can be stably put on the forehead 3 to reduce the body temperature.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. An ice pack comprising a main body, a center of which is a bag fixing part, with that two ends of the bag fixing part are extended respectively with a first fastener and a second fastener and a side of the bag fixing part is connected and fixed to a bag through a hook and loop fastener; an interior of the bag being filled with refrigerant and a side of the bag being provided with a hook and loop fastener which corresponds to the bag fixing part, allowing the bag to be fixed on the bag fixing part of the main body; a bottom at a center of the bag being extended and protruded with at least one positioning part which is positioned on two sides of a nose bridge when the ice pack is used.

2. The ice according to claim 1, wherein the main body assumes a long rectangular unit in shape.

3. The ice pack according to claim 1, wherein a bottom end of the bag is extended with an extension part.

4. The ice pack according to claim 1, wherein the first fastener and the second fastener are hook and loop fasteners, allowing the first fastener and the second fastener to be connected and fixed with each other.

5. The ice pack according to claim 1, wherein the positioning part is provided on a pair basis.
